(19) United States
(12) Patent Application Publication
(10) Pub. No.: US 2010/0255159 A1
(43) Pub. Date: Oct. 7, 2010

(54) Method and System for Making a Consistent Dough Sheet

(75) Inventors: Christopher Adloff, Plano, TX (US); Ruben Enriquez, San Antonio, TX (US); Terry Moros, Frisco, TX (US); Scott Richardson, Gainesuille, TX (US); Billy Mcelheney, Midlothian, TX (US)

Correspondence Address: CARSTENS & CAHOON, LLP, 13760 NOEL ROAD, SUITE 900, DALLAS, TX, 75240, US

(73) Assignee: FRITO-LAY NORTH AMERICA, INC., Plano, TX (US)

(21) Appl. No.: 12/417,980

(22) Filed: Apr. 3, 2009

(51) Int. Cl. A21D 2/02; G01N 33/02; A21C 3/02

(52) U.S. Cl. 426/231; 425/135; 425/141

(57) ABSTRACT

A method and system for making a dough sheet with consistent properties across its width and over time is disclosed. The moisture content of dough is measured using a near-infrared spectrometer. The dough is evenly distributed across the length of sheeting rollers by an externally reinforced fan plate. A control system uses the moisture content and level of dough on the sheeting rollers to control ingredient flow rates.

### BACKGROUND OF THE INVENTION

1. Technical Field

The present invention relates to a method and system for producing a dough sheet with consistent qualities across its width, and over time. In particular, the present invention relates to a method and system for determining, inline, the moisture content of a dough mixture just before it is sheeted, using near-infrared measurements, and using said moisture content, in combination with other variables to control the dough mixing and sheeting process. The present invention also relates to a method and system for improved control of the flow of dough throughout the sheeting process.

2. Description of Related Art

Dough is an important part of the snack food production process. A dough mixture is created by combining a starchy component with water and other ingredients to create a pliable mass. It is known that a dough mixture can be passed through pairs of counter-rotating rollers to create a dough sheet. The qualities of the dough mixture, such as the moisture content, can be measured offline, online or inline.

Offline measurement occurs when a portion of the dough is actually removed from the manufacturing line and subjected to laboratory analysis. Such offline analyses can take anywhere from 3 minutes to 24 hours to complete. Offline analysis of moisture content does not typically allow practitioners to exert adequate control over the dough making and sheeting process.

Online measurements are done at or close to the area of production. Some online measuring techniques still involve removing a portion of the product from the line, but the analysis occurs near the production line. Online measurement provides the opportunity to exercise finer control over the production process than offline measurements.

Inline measurement is done without removing any product from the product line, except perhaps bypassing a portion of the product stream to a measuring device, and then reintroducing the bypass stream into the main product line.

A number of different techniques have been used in the snack food art to measure the moisture content of a dough mixture. Subjecting the dough sheet to nuclear magnetic resonance (NMR) analysis, guided microwave spectrometry (GMS), or impedance/capacitance analysis has been used in the art to determine the moisture content of the dough. Each of these analytical techniques face serious challenges with respect to the typical conditions under which a dough mixture is created and sheeted.

For example, NMR analysis is not tolerant of temperature changes. In order for NMR analysis to yield useful and accurate results, even when combined with impedance/capacitance analysis, the dough must be maintained a constant temperature. Such a measurement condition is completely unrealistic under real-world processing conditions. Similarly, GMS analysis becomes unreliable when manufacturing temperatures fall outside a fairly narrow temperature range. Typical dough processing can easily involve dough temperatures that are above the range in which GMS is an effective analytical technique. All of these methods are also dependent on product density and ingredient particle size, variations in which can further complicate the analysis. In sum, the methods used in the prior art to measure dough temperature do not adequately address the wide variations in processing conditions that occur during the dough making and sheeting process. The present invention allows for very accurate measurement of dough mixture moisture content in a way that overcomes the problems in the prior art, and provides additional advantages with respect to dough processing.

### SUMMARY OF THE INVENTION

In accordance with one aspect of the present invention, there is provided a method of measuring the moisture content of a dough mixture inline. In a preferred embodiment, the dough mixture is a masa dough mixture. The method includes the steps of mixing dough ingredients with water to form the dough mixture, pumping the dough mixture through a fan plate and then towards and through a pair of counter-rotating sheeting rollers. In the preferred embodiment of masa dough, cooked corn and water are combined and mixed in a grinder to create a masa dough. The moisture content of the dough is measured inline just prior to the sheeting step using a near-infrared (NIR) spectrometer.

In one embodiment, the NIR spectrometer impinges the dough with NIR light at an angle of at least ten degrees from normal. In another embodiment, the NIR light is impinged onto the dough at an angle of about 20 degrees from normal.

In one embodiment, the NIR device produces a calculated moisture content of the masa dough which varies less than 0.5% from the actual moisture content of the masa dough at dough temperatures ranging from 95° F. to 110° F. In another embodiment, the NIR device produces a measured moisture content of the masa dough which varies less than 0.5% from the actual moisture content of the masa dough at corn grind sizes ranging from 0.015 inches to 0.025 inches.

In accordance with another aspect of the present invention, the masa dough is pumped through a fan plate before falling onto the sheeting rollers. The fan plate has a narrow opening that is approximately as long as the sheeting rollers. The narrow opening is externally reinforced, in one embodiment, by curved plates.

In accordance with another aspect of the present invention, the level of masa dough on the pair of sheeting rollers is measured, and the measured level is used to control the flow rate of dough mixture through the fan plate.

The above, as well as additional features and advantages of the invention will become apparent in the following written detailed description.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying. drawings, wherein:

FIG. 1 is a flow chart illustrating the processing steps according to one embodiment of the present invention;

FIG. 2 is a profile diagrammatic view of one embodiment of the present invention;

FIG. 3 is a perspective diagrammatic view of one embodiment of the present invention;

Where used in the various figures of the drawing, the same numerals designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the invention.

### DETAILED DESCRIPTION OF THE INVENTION

The present invention relates to a method and system for creating a sheeted dough mixture that has consistent properties upon sheeting and over time. In a preferred embodiment, the dough mixture is a masa dough mixture, which generally comprises a ground corn and water mixture. While the invention will be particularly described with respect to masa dough, it is understood that the principles of this invention can apply to other types of dough (which utilize dry ingredients other than ground corn) and dough mixtures.

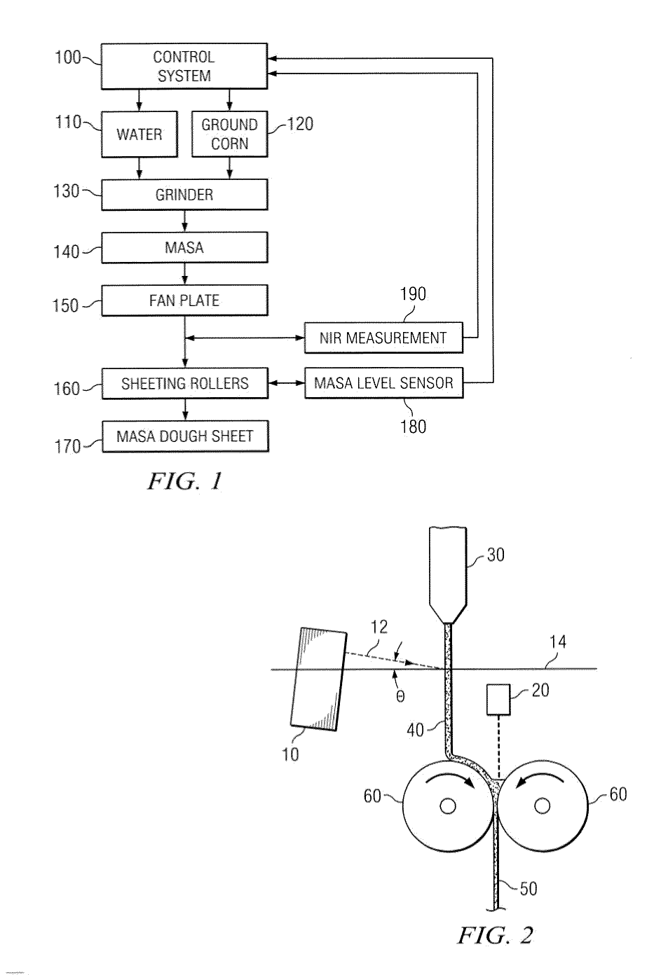

FIG. 1 is a flow chart depicting the processing steps of one embodiment of the present invention. Water 110, corn 120 and, optionally, other ingredients (not shown) are mixed and combined in a grinder 130 to create a masa dough mixture 140. In a preferred embodiment, the grinder comprises two millstones with a gap between them into which the corn and water are fed to make masa dough. The masa dough mixture 140 is then fed through a fan plate 150, which distributes the masa 140 onto a pair of counter-rotating sheeting rollers 160. The masa dough 140 passes through the sheeting rollers 160 to create a masa dough sheet 170.

One of the most important characteristics of the dough mixture as it passes through the sheeting rollers is the dough moisture content. If the moisture content is too high, the dough will stick to the sheeting rollers and produce an inconsistent dough sheet. If the moisture content is too low, the dough will tend to sit above the sheeting rollers and fail to pass through, producing an overworked dough and an inconsistent dough sheet. An inconsistent dough sheet will complicate later processing steps, such as cooking steps, because processing parameters are designed for intermediate dough products with specific, predetermined characteristics, such as thickness, moisture content and weight per ten. In general, the moisture content of the masa dough mixture must be maintained between about 40% and about 60% by weight of the masa dough in order to produce a dough sheet with a consistent thickness, and intermediate dough products with consistent weight per ten. In a preferred embodiment, the moisture content of the masa dough is maintained between about 45% and about 55% by weight.

Referring back to FIG. 1, as the masa dough 140 is being distributed to the sheeting rollers 160, a near-infrared spectrometer impinges the masa dough with electromagnetic radiation at wavelengths in the near-infrared spectrum (typically between about 800 nanometers and 2500 nanometers), and records the absorbance, reflectance or transmittance of the near-infrared light. The NIR spectrometer uses the resulting NIR spectrum to calculate a moisture content for the dough. The moisture content of the dough can be measured by examining the absorbance of NIR light in the 1900 to 2000 nanometer band and comparing it to data collected on known samples. During the sheeting process, NIR measurements 190 are continuously or periodically collected and the calculated moisture contents are calculated and averaged over time. The averaging process provides an accurate measure by reducing errors due to local variations in the masa dough in the area of measurement.

The calculated moisture content 190 is then used by the control system 100 to adjust the relative amounts of corn 120 and/or water 110 being fed into the grinder 130.

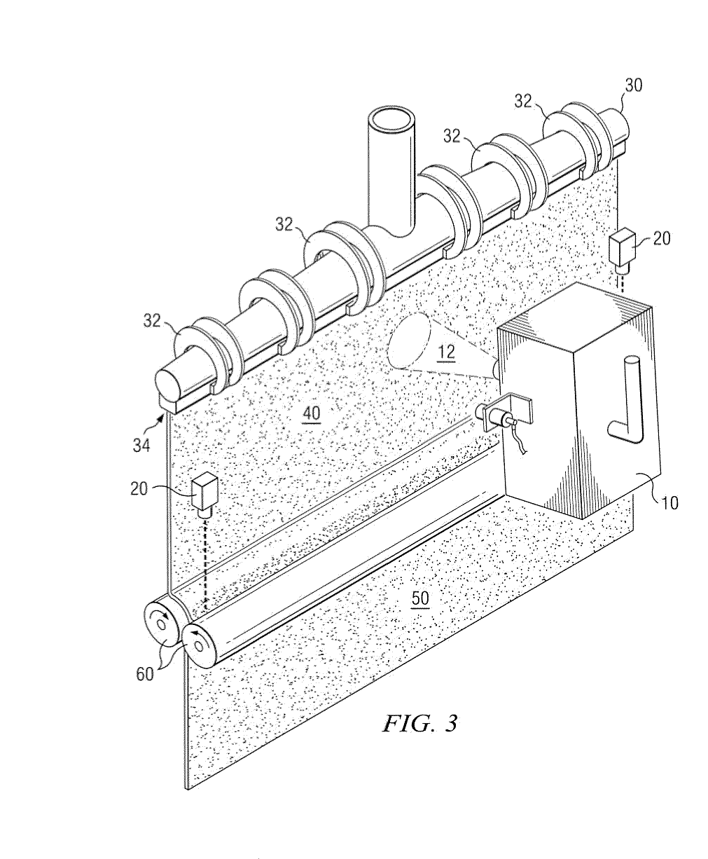

FIGS. 2 and 3 diagrammatically depict one embodiment of the dough sheeting system of the present invention. The masa dough is pumped from the grinder (not shown) through a fan plate 30. The fan plate drops a curtain of masa dough 40 approximately as long as the sheeting rollers 60 onto one of the sheeting rollers 60. The rotation of the sheeting rollers 60 brings the masa dough 40 towards and through the nip between the sheeting rollers 60.

As the masa dough 40 passes from the fan plate 30 towards the sheeting rollers 60, as described above, an NIR spectrometer 10 uses detected near-infrared light 12 to calculate a moisture content of the dough. As such, the moisture content measurement is done inline. It is also done without bypass.

Others have used NIR spectroscopy to measure the moisture contents of pourable, low moisture grain products inline, with bypass. U.S. Pat. No. 5,406,084 shunts a portion of the grain being processed off the main processing line and through all NIR measuring device. The measured grain is then reintroduced into the main product stream by means of a screw conveyor. This bypass setup is not feasible for use with the dough sheeting process of the present invention because of the work input the bypass process imparts into the dough. As described above, moisture content is an important factor in determining the quality and consistency of a dough sheet. Work input is another important factor that affects the consistency and sheetability of the dough. All of the dough across the width and length of the dough sheet needs to have a consistent amount of work input in order to produce a consistent sheet. The work input also affects the thickness and the weight per ten of intermediate dough products. As described above, these properties need to be within specific, known ranges in order for later processing steps to be effective and efficient.

With specific respect to the grain processing patent cited above, the portion of grain that bypasses the main processing line receives a greater amount of work input (mostly by the extra screw conveyor) than the grain that remains in the main processing line. Such discrepancies in work input may not be as important in the processing of a pourable commodity like grain. There is certainly no suggestion in the reference that the bypass arrangement causes any particular problems with grain processing. However, the Applicants herein have determined that such an arrangement, were it to be used with a dough product that is sheeted, would produce widely varying amounts of work input into the dough, and would therefore produce a dough mixture and dough sheet with varying consistency and sheetability.

Applicants' NIR measurement of the masa dough in the present invention also differs from the NIR measurement of low moisture, pourable grain, described above. The relatively higher moisture dough mixture passing out of the fan plate and towards the sheeting rollers has a thin, shiny layer of moisture on its outer surface. As depicted in FIG. 2, Applicants herein have determined that NIR analysis gives the most accurate results when the NIR radiation 12 impinges the dough surface 40 at a non-normal angle θ. A line normal to the dough surface is referred to as line 14. When the NIR radiation impinges the dough sheet at a normal angle, Applicants found that an undesirable “glare” or mirroring effect from the moisture on the surface of the dough caused a wider variation in, and therefore less accurate, calculated moisture content. In a preferred embodiment, the NIR radiation impinges the dough surface at an angle θ of at least 10 degrees from normal line 14. In a most preferred embodiment, the angle θ is approximately 20 degrees from normal line 14.

In another embodiment, the masa dough mixture is pumped through an externally reinforced fan plate 30 in order to evenly distribute it across the length of the sheeting rollers 60. Even distribution of dough across the length of the sheeting rollers is another important factor in producing a dough sheet with consistent properties across its width. The fan plate 30 spreads the masa dough 40 out from a feed pipe and passes it through a narrow opening 34 that is approximately the same length as the length of the sheeting rollers 60. The preferred width of the opening 34 is less than 0.5 inches. Wider openings yield a masa curtain that does not sheet as well, or yield as accurate moisture content readings from the NIR spectrometer.

Fan plates that are known in the art have several drawbacks, which are solved by the present invention. Some fan plates are not reinforced at all across their width. As a result, the opening in the fan plate tends to bow outward near the center of the fan plate, causing more masa dough to be ejected from the center of the fan plate than from the outer edges. This, in turn, causes more dough to pile up in the center of the sheeting rollers, and causes a dough deficiency at the edges of the sheeting rollers. The dough in the center of the sheeting rollers is worked more as it piles up, and the dough sheet can experience thickness near its center, and thinness and holes near its outer edges. Another type of fan plate previously used has an opening width that is reinforced in places along its length, but the reinforcement heretofore has been an internal reinforcement. Such internal reinforcement has come in the form of bolts that span the width of the fan plate opening, preventing the opening from deforming during the sheeting process. The bolts do provide reinforcement for the fan plate, but because they are internal to the fan plate, they impede the flow of masa dough through the fan plate opening at their specific locations. The inconsistent flow of masa dough through the fan plate causes irregularities in the dough as it falls on top of the sheeting rollers, which in turn can cause the sheeting problems described above.

Applicants herein have developed an external reinforcement system for a masa dough fan plate 30 that reduces or eliminates the problems associated with previous fan plates. The particular external reinforcements for one embodiment of the present invention comprise one or more curved support plates 32, each affixed to both sides of the fan plate 30. Although the particular embodiments depicted in FIG. 3 are rounded or curved supports, support plates 32 having other shapes would also work within the teachings of the present invention. The important overarching principle is that the supports 32 are affixed to the exterior of the fan plate 30, and that the bolts or other means for attaching them do not protrude into the interior of the fan plate where the masa dough mixture is flowing.

This externally reinforced fan plate 30 provides a thinner, more pliable and more evenly distributed wall of masa dough 40 to the sheeting rollers 60 than is available in the prior art. The lack of impedance to masa dough flow also provides a smooth surface for the NIR measurement described above. The smoother surface allows the NIR spectrometer to calculate more accurate dough moisture contents than otherwise possible.

Another important aspect of the dough sheeting system and method is the ability to control the level of dough piling up on the sheeting rollers. In one embodiment, a level sensor 20 that detects the level of masa dough 40 above the nip between the sheeting rollers 60 is provided. In a preferred embodiment, more than one level sensor 20 is provided to detect the masa dough level at more than one location across the length of the sheeting rollers. Level sensors that work with the present invention include laser sensors and ultrasonic sensors. In another preferred embodiment, the masa dough 40 is pumped from the fan plate 30 onto one of the sheeting rollers 60, rather than straight onto the nip between the rollers. This arrangement prevents or reduces dough pileup and maintains the sheet-like properties and laminar flow of the masa dough 40 exiting the fan plate 30.

Referring back to FIG. 1, the detected dough level 180 is then used by the control system 100 to adjust the amount of water 110 and corn 120 being fed into the grinder 130 and the flow rate of the masa dough through the fan plate. Keeping the dough level on the sheeting rollers within a specific range prevents overworking and short sheeting of the dough, and helps the consistency of the dough by maintaining the dough's laminar flow from the fan plate through the sheeting rollers. In sum, the moisture content reading 190 from die NIR spectrometer is used by the control system 100 to control the relative flow rates of corn 120 and water I 10, while the level sensor 180 is used by the control system 100 to control the overall flow rates of corn 120 and water 110.

The combination of the above features of the dough sheeting system of the present invention dramatically improves the consistency of the dough sheet across its width and across the entire sheet over time under typical manufacturing conditions. The increases in consistency are seen in consistent dough thickness exiting the sheeting rollers, and a consistent weight per ten of the intermediate dough products. The final products obtained following the sheeting method of the present invention are also more consistent because subsequent processing steps are designed for known and predetermined intermediate properties, and the present process consistently provides intermediate products with properties in the predetermined ranges.

In particular, the method and system of the present invention can calculate the moisture content of the masa dough exiting the fan plate to within 0.5% of the actual moisture content over a temperature range of about 95° F. to about 110° F., and over a corn grind size of about 0.012 inches to about 0.025 inches. As used herein, the term “corn grind size” refers to the size of the gap between the two millstones which comprise the grinder through which the corn has passed. Such accuracy is unknown and unattainable in the prior art over these temperature ranges. The system will also yield useful, yet slightly less accurate, results at temperatures from 80° F. to 120° F., and corn grind sizes from 0.008 inches to 0.03 inches.

While the invention has been particularly shown and described with reference to several embodiments, it will be understood by those skilled in the art that various other approaches and applications to industry may be made without departing from the spirit and scope of the invention disclosed herein.

We claim:

1. A method of making a dough sheet, said method comprising:
mixing dry ingredients and water to create a dough having an actual moisture content;
calculating a calculated moisture content corresponding to said actual moisture content for said dough by measuring absorbance, transmittance, or reflectance of near-infrared light by said dough, inline, without bypass;
maintaining said actual moisture content within a predetermined range based on said calculating; and
passing said dough between at least one pair of sheeting rollers to make said dough sheet.

2. The method of claim 1 wherein said actual moisture content is between about 40% and about 60%.

3. The method of claim 1 wherein said actual moisture content is between about 45% and about 55%.

4. The method of claim 1 wherein said measuring occurs immediately preceding said passing.

5. The method of claim 1 further comprising after said mixing:
distributing said dough onto said sheeting rollers through an externally reinforced fan plate, and wherein said measuring occurs immediately after said distributing.

6. The method of claim 1 wherein said measuring further comprises
impinging said dough with electromagnetic radiation in the near-infrared spectum at a non-normal angle.

7. The method of claim 1 further comprising:
measuring a level of said dough on said sheeting rollers; and
maintaining said level within a predetermined range.

8. The method of claim 1 wherein said calculated moisture content is within 0.5% of said actual moisture content for dough temperatures ranging from about 95° F. to about 110° F.

9. The method of claim 1 wherein said dough is masa dough, and said calculated moisture content is within 0.5% of said actual moisture content for corn grind sizes ranging from 0.012 inches to 0.025 inches.

10. The method of claim 1 wherein said dough is masa dough, and said dry ingredients comprise corn, and wherein said mixing occurs in a grinder.

11. The method of claim 1 wherein said calculating occurs at a dough temperature between about 80° F. and 120° F.

12. The method of claim 10 wherein said calculating occurs at corn grind sizes between about 0.008 inches and about 0.03 inches.

13. A system for producing a dough sheet comprising:
a continuous mixer for mixing dry ingredients with water to produce a dough having an actual moisture content;
one pair of sheeting rollers to produce a dough sheet from dough received from said continuous mixer;
a near-infrared spectrometer adapted to measure absorbance, reflectance or transmittance of near-infrared light by said dough before said dough is received by said sheeting rollers, and calculating a calculated moisture content corresponding to said actual moisture content for said dough, inline, without bypass;
a control system to maintain said actual moisture content within a predetermined ranges based on said calculated moisture content.

14. The system of claim 13 further comprising:
a fan plate having at least one external reinforcement adapted to distribute said dough to said sheeting rollers.

15. The system of claim 13 further comprising:
at least one level sensor adapted to measure a dough level on said sheeting rollers, and wherein said control system maintains said dough level within a predetermined range.

16. The system of claim 13 wherein said near-infrared spectrometer is adapted to impinge said near-infrared light onto said dough at a non-normal angle.

17. The system of claim 16 wherein said non-normal angle is at least 10 degrees.

18. The system of claim 14 wherein said at least one external reinforcement comprises at least one curried plate attached to two sides of said fan plate.

19. The system of claim 13 wherein said calculated moisture content is within 0.5% of said actual moisture content for dough temperatures ranging from about 95° F. to about 110° F.

20. The system of claim 13 wherein said dough is masa dough, and said calculated moisture content is within 0.5% of said actual moisture content for corn grind sizes ranging from 0.012 inches to 0.025 inches.

21. The system of claim 13 wherein said dough is masa dough, and wherein said dry ingredients comprise corn, and wherein said continuous mixer comprises a grinder.

22. The system of claim 13 wherein said near-infrared spectrometer is further adapted to operate at dough temperatures between about 80° F. and about 120° F.

23. The system of claim 21 wherein said near-infrared spectrometer is further adapted to operate at corn grind sizes between about 0.008 inches and 0.03 inches.
